(19) United States
(12) Patent Application Publication
(10) Pub. No.: US 2026/0004924 A1
(43) Pub. Date: Jan. 1, 2026

(54) SYSTEM FOR MONITORING PATIENTS IN A MEDICAL FACILITY

(71) Applicant: TERUMO KABUSHIKI KAISHA, Tokyo (JP)

(72) Inventors: Yoshihito MACHIDA, Sagamihara Kanagawa (JP); Arisa YOSHIKAWA, Shanghai (CN)

(21) Appl. No.: 19/322,523

(22) Filed: Sep. 8, 2025

Related U.S. Application Data
(63) Continuation of application No. PCT/JP2024/008354, filed on Mar. 5, 2024.

(30) Foreign Application Priority Data
Mar. 9, 2023 (JP) 2023-036901

(51) Int. Cl. G16H 40/20; A61B 5/00; G16H 10/60; G16H 50/20

(52) U.S. Cl. CPC G16H 40/20; A61B 5/742; A61B 5/746; G16H 10/60; G16H 50/20

(57) ABSTRACT

A system for monitoring patients in a medical facility includes a circuit connectable to a server, a display, an input device, and a processor configured to: generate a first screen related to one of the patients and accepting: a condition for an alert to be output depending on a value corresponding to a physiological parameter of the patient, and a type of the alert, display the first screen, and after a first condition for a first physiological parameter and a first type are input for a first patient, generate a second screen showing the patients and capable of outputting an alert for each patient, display the second screen, acquire a first value corresponding to the first parameter from the server, and upon determining that the first condition is met, update the second screen to output an alert of the first type in association with the first patient.

### CROSS-REFERENCE TO RELATED APPLICATION

This application is a continuation of International Patent Application No. PCT/JP2024/008354 filed March 5, 2024, which is based upon and claims the benefit of priority from Japanese Patent Application No. 2023-0036901, filed March 9, 2023, the entire contents of which are incorporated herein by reference.

### BACKGROUND

### TECHNICAL FIELD

The present disclosure relates to a system for monitoring patients in a medical facility.

### Related Art

Existing systems can display patients in a waiting room or emergency room as icons, color-coded based on their triage and Sequential Organ Failure Assessment (SOFA) scores.

In a large medical facility that manages numerous patients, it is essential to quickly identify individuals who require urgent care. However, patients have diverse attributes, such as age or underlying medical conditions, making it difficult to identify those in greatest need of care based solely on standardized metrics like triage or SOFA scores.

### SUMMARY

Embodiments of the present disclosure enable quick identification of patients in greater need of care.

An aspect of the present disclosure provides a system for monitoring patients in a medical facility, the system comprising: an interface circuit connectable to a server that stores patient information about each of the patients, the patient information including a value corresponding to each of one or more physiological parameters of the patient; a display; an input device; a memory that stores a program; and a processor configured to execute the program to: generate a first screen related to one of the patients and accepting an input of: a condition for an alert to be output depending on a value corresponding to a physiological parameter of said one of the patients, and a type of the alert, control the display to display the generated first screen, and after a first condition for a first physiological parameter and a first type are input for a first patient via the input device, generate a second screen showing a list of the patients and capable of outputting an alert for each of the patients, control the display to display the generated second screen, acquire a first value corresponding to the first physiological parameter from the server, determine whether the first condition is met based on the first condition and the acquired first value, and upon determining that the first condition is met, update the second screen to output an alert of the first type in association with the first patient.

With this configuration, the system quickly identifies patients who need medical attention.

### BRIEF DESCRIPTION OF DRAWINGS

FIG. 1 is a diagram illustrating a configuration of an integrated system according to an embodiment.

FIG. 2 is a block diagram illustrating a configuration of a healthcare worker terminal of the integrated system.

FIG. 3 is a flowchart illustrating operation of the healthcare worker terminal.

FIG. 4 is a diagram illustrating an example of a screen on the healthcare worker terminal.

FIG. 5 is a flowchart illustrating the operation of the healthcare worker terminal.

FIG. 6 is a diagram illustrating an example of the screen on the healthcare worker terminal.

FIG. 7 is a diagram illustrating an example of the screen on the healthcare worker terminal.

### DETAILED DESCRIPTION

Hereinafter, embodiments of the present disclosure will be described with reference to the drawings.

In the drawings, the same or corresponding parts or elements are denoted by the same reference numerals. In the description, explanations for the same parts or elements will be omitted or simplified as appropriate.

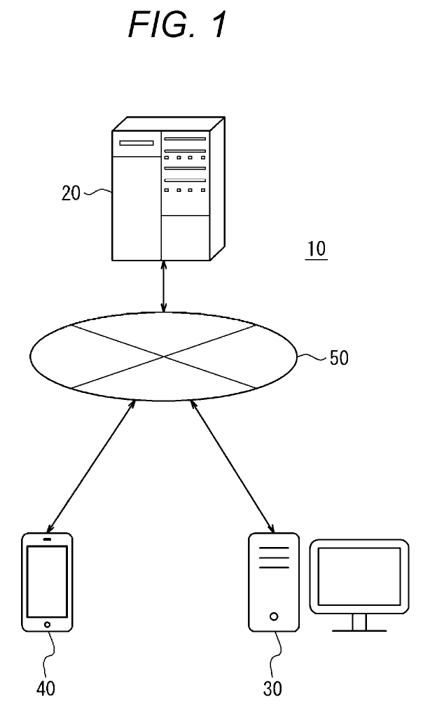

A configuration of an integrated system 10 according to an embodiment will be described with reference to FIG. 1.

The integrated system 10 includes a server 20, a healthcare worker terminal 30, and a patient terminal 40. The server 20 is configured to communicate with the healthcare worker terminal 30 and the patient terminal 40 via a network 50. The healthcare worker terminal 30 may be configured to communicate with the patient terminal 40 via the network 50.

The server 20 is a computer belonging to a cloud computing system or another computing system installed in a data center or similar facility. The server 20 is operated by an operator offering services for storing patient information.

The patient information includes values of at least two patient observation tems. Examples of the values of observation items include input values related to patient's physical information, such as weight. The physical information is information regarding the body, and includes information regarding weight, height, or abdominal circumference. Other examples of the values of observation items include input values related to patient's vital sign information, such as blood pressure or pulse rate. The vital sign information is information regarding a sign indicating that a human is in a living state, and includes information regarding blood pressure, pulse rate, body temperature, and respiration rate. Still other examples of the values of observation items include input values related to patient's symptoms, such as presence or absence of dyspnea, edema, fatigue, loss of appetite, or insomnia. Further examples of the values of observation items include patient's physical measurements obtained using a measuring device, such as a weighing scale, a blood pressure monitor, or a pulse monitor. That is, the patient information can include the patient's physical information and vital sign information, the input values related to patient's symptoms, and/or the patient's physical measurements. The observation item can be referred to as a physiological parameter or a health indicator.

The healthcare worker terminal 30 is a personal computer (PC) installed in clinics or hospitals to which healthcare workers such as physicians belong. Alternatively, the healthcare worker terminal 30 may be a mobile terminal, such as a mobile phone, a smartphone, or a tablet operated by healthcare workers.

The patient terminal 40 is a mobile terminal, such as a mobile phone, a smartphone, or a tablet owned by patients suffering from heart failure or other illnesses. Alternatively, the patient terminal 40 may be a general-purpose terminal, such as a PC installed in a patient's home.

The network 50 includes the Internet, at least one wide area network (WAN), at least one metropolitan area network (MAN), or any combination thereof. The network 50 may include at least one wireless network, at least one optical network, or any combination thereof. The wireless network is, for example, an ad hoc network, a cellular network, a wireless local area network (LAN), a satellite communication network, or a terrestrial microwave network.

First, an overview of the integrated system 10 will be described.

The healthcare worker terminal 30 accepts an input operation to set an alert application condition C[i,j] and an alert type T[i,j] for each combination of a patient Yi and an observation item Zj via an input device. The alert application condition C[i,j] is a condition for applying an alert for the combination of the patient Yi and the observation item Zj. The alert type T[i,j] is a type of alert applied when the alert application condition C[i,j] is satisfied.

The healthcare worker terminal 30 retrieves the value of each observation item for each patient from the server 20 via the network 50. In an embodiment, the value of each observation item for each patient is uploaded from the patient terminal 40 to the server 20, and is stored linked to or in association with identification information used to identify each patient in a storage provided in the server 20 or a storage provided in another device accessible by the server 20 via the network 50. As a modification, the healthcare worker terminal 30 may directly retrieve the value of each observation item for each patient from the patient terminal 40. The healthcare worker terminal 30 may act as the server 20. The healthcare worker terminal 30 may also act as the server 20.

When determining that the retrieved value of at least one observation item Zb for at least one patient Ya satisfies an alert application condition C[a,b], the healthcare worker terminal 30 displays an alert of alert type T[a, b] on a display as an alert corresponding to the at least one patient Ya together with the identification information used to identify each patient. The alert application condition C[a,b] is a condition for applying an alert set for a combination of the at least one patient Ya and the at least one observation item Zb. The alert type T[a,b] is a type of alert set for the combination of the at least one patient Ya and the at least one observation item Zb.

With this configuration, the alert application condition can be set individually for each patient rather than uniformly. Displaying the alert set as described above enables quick identification of patients who truly require care.

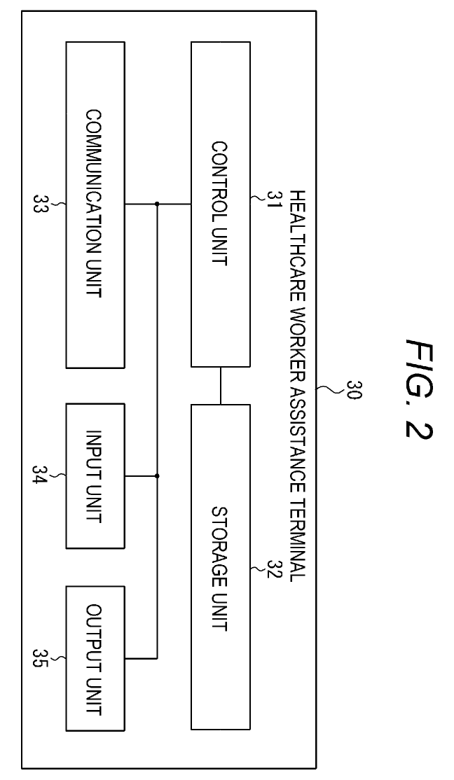

A configuration of the healthcare worker terminal 30 will be described with reference to FIG. 2.

The healthcare worker terminal 30 includes a control unit 31, a storage unit 32, a communication unit 33, an input unit 34, and an output unit 35.

The control unit 31 includes at least one processor, at least one programmable circuit, at least one dedicated circuit, or any combination thereof. The processor is a general-purpose processor such as a central processing unit (CPU) or graphics processing unit (GPU), or a dedicated processor specialized for specific processing. The programmable circuit is, for example, a field-programmable gate array (FPGA). The dedicated circuit is, for example, an application specific integrated circuit (ASIC). The control unit 31 executes processing related to the operation of the healthcare worker terminal 30 while controlling each unit of the healthcare worker terminal 30.

The storage unit 32 includes at least one semiconductor memory, at least one magnetic memory, at least one optical memory, or any combination thereof. The semiconductor memory is, for example, a random access memory (RAM), a read only memory (ROM), or a flash memory. The RAM is, for example, a static random access memory (SRAM) or a dynamic random access memory (DRAM). The ROM is, for example, an electrically erasable programmable read only memory (EEPROM). The flash memory is, for example, a solid-state drive (SSD). "The magnetic memory is, for example, a hard disk drive (HDD). The storage unit 32 functions as, for example, a main memory, an auxiliary storage device, and/or a cache memory. The storage unit 32 stores data used for the operation of the healthcare worker terminal 30 and data obtained through the operation of the healthcare worker terminal 30.

The communication unit 33 includes at least one communication module. The communication module is a network interface circuit compatible with, for example, a wired LAN communication standard such as Ethernet (registered trademark), a wireless LAN communication standard such as IEEE 802.11, or a mobile communication standard such as Long Term Evolution (LTE), 4G, or 5G. "IEEE" is an abbreviation for Institute of Electrical and Electronics Engineers. "4G" is an abbreviation for 4th generation. "5G" is an abbreviation for 5th generation. The communication unit 33 communicates with the server 20. The communication unit 33 may communicate with the patient terminal 40. The communication unit 33 receives data used for the operation of the healthcare worker terminal 30 and transmits data obtained through the operation of the healthcare worker terminal 30.

The input unit 34 includes at least one input device. The input device includes, for example, a physical key, a capacitive key, a pointing device, a touch screen integrated with a display, a camera, and/or a microphone. The input unit 34 accepts an operation to input data to be used for the operation of the healthcare worker terminal 30. The input unit 34 may be connected to the healthcare worker terminal 30 as an external input device instead of being provided in the healthcare worker terminal 30. As a connection interface, an interface compatible with standards such as USB, HDMI (registered trademark), or Bluetooth (registered trademark) can be used. "USB" is an abbreviation for Universal Serial Bus. "HDMI" (registered trademark) is an abbreviation for High-Definition Multimedia Interface.

The output unit 35 includes at least one output device. The output device includes, for example, a display, a printer, and/or a speaker. The display is, for example, a liquid crystal display (LCD) or an organic electro luminescent (EL) display. The output unit 35 outputs data obtained through the operation of the healthcare worker terminal 30. The output unit 35 may be connected to the healthcare worker terminal 30 as an external output device instead of being provided in the healthcare worker terminal 30. As a connection interface, an interface compatible with standards such as USB, HDMI (registered trademark), or Bluetooth (registered trademark) can be used.

The functions of the healthcare worker terminal 30 are executed by its processor or the control unit 31 according to a healthcare worker assistance program. That is, the functions of the healthcare worker terminal 30 are implemented by software. The healthcare worker assistance program enables a computer to function as the healthcare worker terminal 30 by causing the computer to execute the operation of the healthcare worker terminal 30. That is, the computer functions as the healthcare worker terminal 30 by executing the operation of the healthcare worker terminal 30 in accordance with the healthcare worker assistance program.

The program can be stored in a non-transitory computer-readable storage or recording medium. The non-transitory computer-readable medium is, for example, a flash memory, a magnetic recording device, an optical disc, a magneto-optical recording medium, or a ROM. The distribution of the program is carried out, for example, by selling, transferring, or lending a portable medium such as a Secure Digital (SD) card, a digital versatile disc (DVD), or a compact disc read only memory (CD-ROM) that stores the program. The program may be distributed by storing the program in a storage of a server and transferring the program from the server to another computer. The program may be provided as a program product.

The computer temporarily stores, for example, the program stored in the portable medium or the program transferred from the server in the main memory. Then, the computer causes the processor to read the program stored in the main memory and execute processing in accordance with the read program. The computer may read the program directly from the portable medium and execute the processing in accordance with the program. Each time the program is transferred from the server to the computer, the computer may sequentially execute processing in accordance with the received program. The processing may be executed via a so-called ASP-type service that implements functions solely through execution instructions and result retrieval without transferring the program from the server to the computer. "ASP" is an abbreviation for application service provider. The program includes information intended for use in processing by an electronic computer and equivalent to the program. For example, data that is not a direct command to the computer but has a property that defines processing of the computer corresponds to "information equivalent to the program".

Some or all of the functions of the healthcare worker terminal 30 may be implemented by a programmable circuit or dedicated circuit serving as the control unit 31. That is, some or all of the functions of the healthcare worker terminal 30 may be implemented by hardware, e.g., circuitry or one or more circuits.

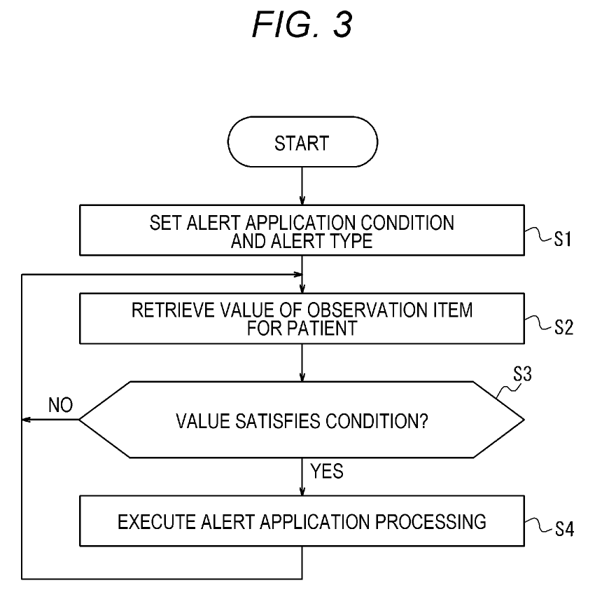

The operation of the healthcare worker terminal 30 will be described with reference to FIG. 3. This operation is also referred to as a healthcare worker assistance method.

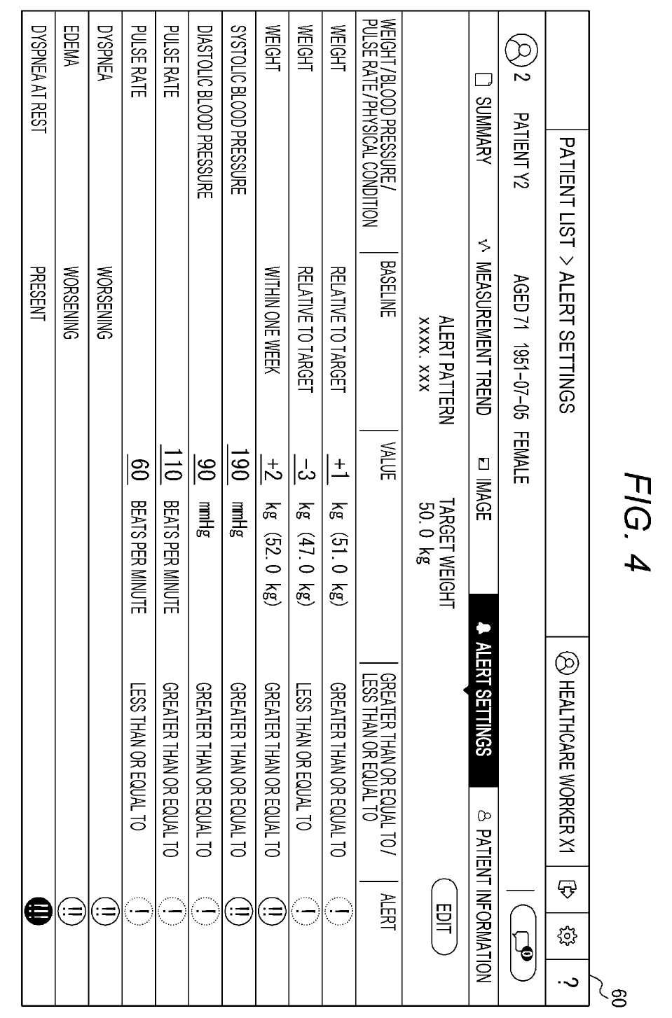

In Step Si, the control unit 31 detects and accepts an input operation to set the alert application condition C[i,j] and the alert type T[i,j] for each combination of the patient Yi and the observation item Zj via the input unit 34. The control unit 31 can generate alert information that associates the patient Yi, the observation item Zj, the alert application condition C[i,j], and the alert type T[i,j], and store the generated information in the storage unit 32. For example, the control unit 31 generates and displays an alert setting screen 60 as illustrated in FIG. 4 on the display serving as the output unit 35 in response to an operation to select a patient Y2. On the alert setting screen 60, alert settings related to various observation items such as physical information and vital sign information of the patient Y2 at home and subjective symptoms of the patient Y2 are displayed together with identification information used to identify the patient Y2 and attribute information such as the age and gender of the patient Y2. On the alert setting screen 60, the alert settings related to each observation item can be changed.

In the example illustrated in FIG. 4, when an increase in weight relative tothe target is set as an observation item Z1, for a combination of the patient Y2 and the observation item Z1, an alert application condition C[2,1] is set such that the alert is applied when the increase in weight relative to the target is greater than or equal to 1 kg, and an alert type T[2,1] is set to "caution". The display color of an icon indicating the alert type "caution" may be set to a desired color such as white. When a decrease in weight relative to the target is set as an observation item Z2, an alert application condition C[2,2] is set for a combination of the patient Y2 and the observation item Z2 such that the alert is applied when the decrease in weight relative to the target weight is greater than or equal to 3 kg, and an alert type T[2,2] is set to "caution". When an increase in weight within one week is set as an observation item Z3, an alert application condition C[2,3] is set for a combination of the patient Y2 and the observation item Z3 such that the alert is applied when the increase in weight within one week is greater than or equal to 2 kg, and an alert type T[2,3] is set to "warning". The type "warning" indicates a higher level of risk than the type "caution". The display color of an icon indicating the alert type "warning" may be set to a particular color such as yellow.

In the example illustrated in FIG. 4 , when systolic blood pressure is set as an observation item Z4, an alert application condition C[2,4] is set for a combination of the patient Y2 and the observation item Z4 such that the alert is applied when the systolic blood pressure is greater than or equal to 190 mmHg, and an alert type T[2,4] is set to "warning". When diastolic blood pressure is set as an observation item Z5, an alert application condition C[2,5] is set for a combination of the patient Y2 and the observation item Z5 such that the alert is applied when the diastolic blood pressure is greater than or equal to 90 mmHg, and an alert type T[2,5] is set to "caution".

In the example illustrated in FIG. 4, when tachycardia is set as an observation item Z6, an alert application condition C[2,6] is set for a combination of the patient Y2 and the observation item Z6 such that the alert is applied when the pulse rate is greater than or equal to 110 beats per minute, and an alert type T[2,6] is set to "caution". When bradycardia is set as an observation item Z7, an alert application condition C[2,7] is set for a combination of the patient Y2 and the observation item Z7 such that the alert is applied when the pulse rate is less than or equal to 60 beats per minute, and an alert type T[2,7] is set to "caution".

In the example illustrated in FIG. 4, when dyspnea is set as an observation item Z8, an alert application condition C[2,8] is set for a combination of the patient Y2 and the observation item Z8 such that the alert is applied when dyspnea worsens, and an alert type T[2,8] is set to "warning". When edema is set as an observation item Z9, an alert application condition C[2,9] is set for a combination of the patient Y2 and the observation item Z9 such that the alert is applied when edema worsens, and an alert type T[2,9] is set to "warning". When dyspnea at rest is set as an observation item Z10, an alert application condition C[2,10] is set for a combination of the patient Y2 and the observation item Z10 such that the alert is applied when dyspnea at rest is present, and an alert type T[2,10] is set to "critical". The type "critical" indicates a higher level of risk than the type "warning". The display color of an icon indicating the alert type "critical" may be set to a particular color such as red.

In step S2, the control unit 31 retrieves the value of the at least one observation item Zb for the at least one patient Ya from the server 20 via the communication unit 33. For example, the control unit 31 transmits a request including the identification information used to identify the patient Y2 to the server 20 via the communication unit 33. Upon receipt of the request from the healthcare worker terminal 30, the server 20 retrieves the values of the observation items Z1,..., and Z10 for the patient Y2 stored in the storage linked to or in association with the identification information included in the received request. The server 20 transmits a response including the identification information used to identify the patient Y2 and the retrieved values of the observation items Z1,..., and Z10 for the patient Y2 to the healthcare worker terminal 30. The control unit 31 retrieves the values of the observation items Z1,..., and Z10 for the patient Y2 by receiving the response from the server 20 via the communication unit 33.

In step S3, the control unit 31 determines whether the value of the at least one observation item Zb for the at least one patient Ya retrieved in step S2 satisfies the alert application condition C[a,b]. For example, assuming that the values of the observation items Z1,..., and Z10 for the patient Y2 have been retrieved in step S2, the control unit 31 determines whether the values of the observation items Z1,..., and Z10 for the patient Y2 satisfy the alert application conditions C[2,1],..., and C[2,10] set for the combinations of the patient Y2 and the observation items Z1,..., and Z10 in step S1, respectively.

In a case where it is determined in step S3 that the value of the at least one observation item Zb for the at least one patient Ya satisfies the alert application condition C[a,b], the process in step S4 is executed. On the other hand, in a case where it is determined in step S3 that the value of the at least one observation item Zb for the at least one patient Ya does not satisfy the alert application condition C[a,b], the process in step S2 is repeated.

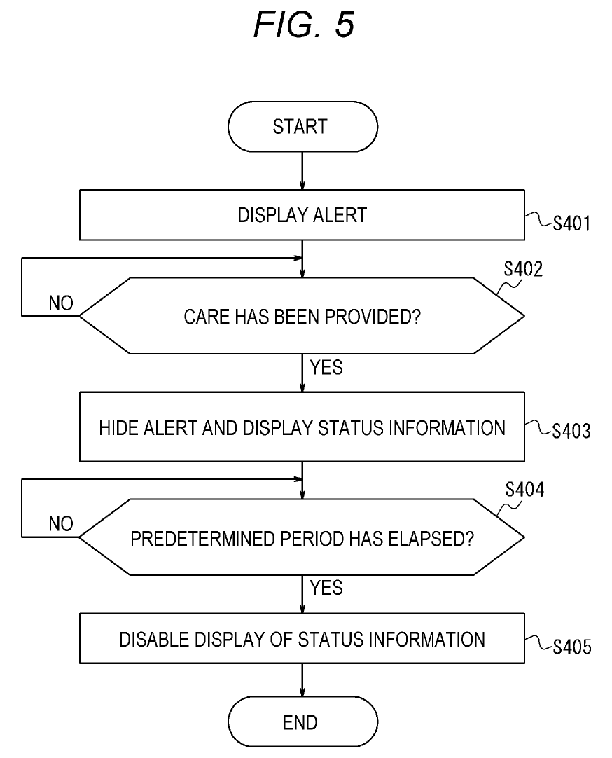

In step S4, the control unit 31 executes alert application processing. A specific procedure of the alert application processing is illustrated in FIG. 5.

In step S401, the control unit 31 displays the alert of alert type T[a,b] as the alert corresponding to the at least one patient Ya on the display together with the identification information used to identify each patient. For example, the control unit 31 color-codes the displayed alerts on the basis of their respective alert types T[a,b]. The control unit 31 further displays information regarding the value of the at least one observation item Zb for the at least one patient Ya in the same color as the displayed alert.

In step S402, the control unit 31 determines whether the healthcare worker has provided care to the at least one patient Ya while continuing displaying the alert started in step 5401. Upon acceptance of an operation to input, via the input unit 34, care provided information indicating that care has been provided to the at least one patient Ya, the control unit 31 determines that care has been provided. For example, until the operation to input the care provided information is accepted, the control unit 31 continues displaying the alert started in step S401, and displays care not provided information indicating that care has yet to be provided on the display together with the identification information used to identify each patient.

The process in step S402 is repeated until it is determined in step S402 that care has been provided. When it is determined in step S402 that care has been provided, the process in step S403 is executed.

In step S403, the control unit 31 hides or disables the alert started in stepS401, and displays status information indicating that care has been provided on the display together with the identification information used to identify each patient. For example, the status information includes information indicating the time elapsed since care was provided. The display of the care not provided information is replaced with the display of the status information.

In step S404, the control unit 31 determines whether a predetermined period P has elapsed from the start of the display of the status information while continuing displaying the status information started in step S403. The period P is a fixed period in an embodiment, but may be a period configured to vary in a manner that depends on the attribute of the at least one patient Ya such as age or disease, or may be a period configured to vary in a manner that depends on the alert type T[a,b].

The process in step S404 is repeated until it is determined in step S404that the period P has elapsed. When it is determined in step S404 that the period P has elapsed, the process in step S405 is executed.

In step S405, the control unit 31 disables the display of the status information started in step S403. That is, the control unit 31 continues displaying the status information for the period P and does not display the status information after the period P has elapsed.

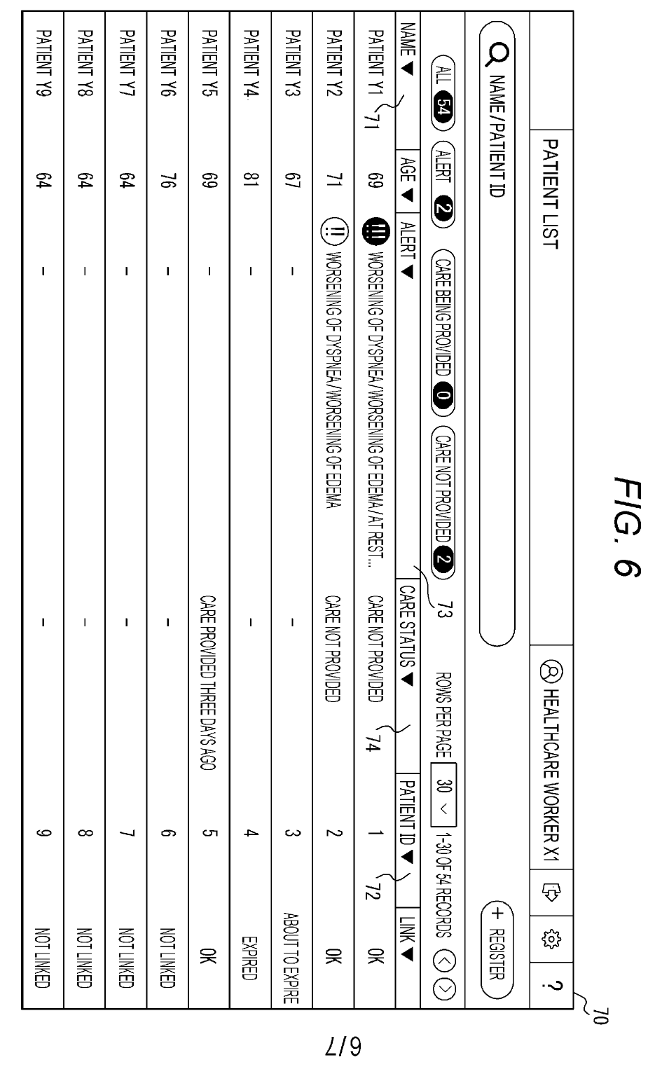

For example, the control unit 31 generates and displays a patient list screen 70 as illustrated in FIG. 6 on the display. On the patient list screen 70, at least a name 71, a patient ID 72, an alert mark 73, and a care status 74 are displayed for each patient. "ID" is an abbreviation for identifier. The patient ID 72 corresponds to the identification information used to identify each patient. As the alert mark 73, the alert type "critical" is represented by a red circle marked with white symbols "!! !", the alert type "warning" is represented by a yellow circle marked with black symbols "! !", and the alert type "caution" is represented by a white circle marked with a black symbol "!". The care status 74 corresponds to the care not provided information or the status information.

In the example illustrated in FIG. 6, the values of the observation items Z8and Z9 for the patient Y2 satisfy the alert application conditions C[2,8] and C[2,9], respectively. Assuming that the example illustrated in FIG. 4 is applied, since the alert types T[2,8] and T[2,9] are set to "warning", the yellow circle marked with black symbols "! !" representing the alert type "warning" is displayed as the alert mark 73 in the row corresponding to the patient Y2. Next to the alert mark 73, information regarding the values of the observation items Z8 and Z9 for the patient Y2, "worsening of dyspnea/worsening of edema", is further displayed in text of the same color as the alert mark 73. Neither a healthcare worker X1 who is the user of the healthcare worker terminal 30 nor another healthcare worker has provided care to the patient Y2. Therefore, in the row corresponding to the patient Y2, the care not provided information "care not provided" is displayed in text as the care status 74. Until care is provided to the patient Y2, the yellow circle marked with black symbols "! !" indicating the alert type "warning" and the care not provided information "care not provided" continue being displayed. The row corresponding to the patient Y1 is substantially the same as the row corresponding to the patient Y2, but the value of the observation item Z10 for the patient Y1 satisfies the alert application condition C[1,10], so the red circle marked with white symbols "!!!" indicating the alert type "critical" is displayed as the alert mark 73 in the row corresponding to the patient Y1.

In the example illustrated in FIG. 6, none of the values of observation items for a patient Y3 satisfy their respective alert application conditions. Therefore, in the row corresponding to the patient Y3, neither the alert mark 73 nor the care status 74 is displayed. The rows corresponding to patients Y4, Y6, Y7, Y8, and Y9 are similar to the row corresponding to the patient Y3; therefore, their explanations will be omitted.

In the example illustrated in FIG. 6, the value of at least one observation item for the patient Y5 satisfied the corresponding alert application condition more than three days ago, but the healthcare worker X1 or another healthcare worker provided care to the patient Y5 three days ago. Therefore, in the row corresponding to the patient Y5, no alert mark 73 is displayed, but the status information (care provided three days ago) is displayed as the care status 74. According to this example, the healthcare worker X1 can easily confirm that "three days" have elapsed since the care was provided. Assuming that the period P is set to one week, the display of the status information is disabled in four days. Specific examples of the care provided to the patient Y5 include admitting the patient Y5 to a hospital, examining the patient Y5, or contacting the patient Y5 to confirm his/her condition directly. Such specific care can be input and displayed.

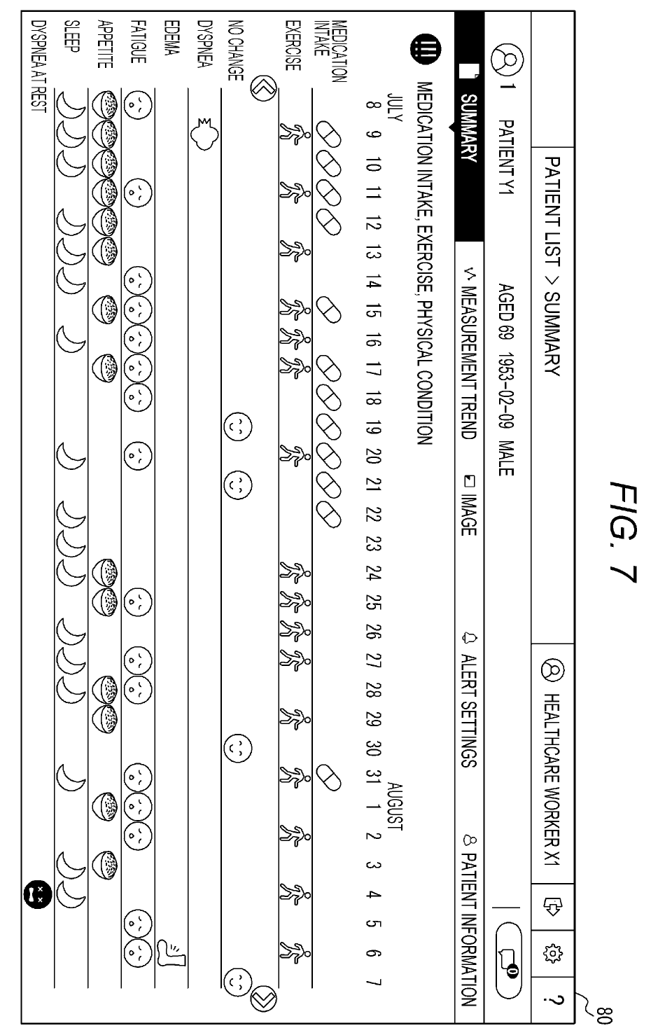

For example, the control unit 31 can generate and display a summary screen 80 as illustrated in FIG. 7 on the display in response to an action to select the patient Y1 as the patient requiring care on the patient list screen 70. On the summary screen 80, icons representing symptoms of the patient Y1 such as dyspnea, edema, fatigue, loss of appetite, lack of sleep, and dyspnea at rest are displayed for each date. The icon of an item requiring care that has entered a critical state is displayed in yellow or red. Its color may be linked to the condition of the patient Y1 or the care status. In the example illustrated in FIG. 7, dyspnea at rest occurred on August 4, so a red icon that corresponds to the color of the case where the alert mark 73 indicates "critical" in the example illustrated in FIG. 6 is arranged at a location corresponding to the combination of August 4 and dyspnea at rest. According to this example, the healthcare worker X1 can easily confirm the cause of the alert in a short time.

The processes from step S2 to step S4 are repeated. Some of the processes may be executed in parallel. For example, after the process in step S401 is executed, the process in step S2 may be executed again in parallel with the processes in step S402 and subsequent steps.

In the above-described embodiments, it is possible to set the levels of the critical state using the alert setting screen 60 as illustrated in FIG. 4. It is possible to display the critical states set for alert-targeted patients using the patient list screen 70 as illustrated in FIG. 6. The targeted patients may be displayed in the upper part of the patient list screen 70. The patient list screen 70 may be sortable on the basis of the patients' critical states. Icons indicating, for example, patients' physical conditions may be displayed in list form using the summary screen 80 as illustrated in FIG. 7. The icon indicating the critical state may be displayed in a different color from the other icons.

According to the above-described embodiments, it is possible to easily identify patients requiring care and easily confirm care status in response to alerts. Therefore, it is possible to improve the efficiency of medical examination and treatment provided by healthcare workers.

Furthermore, according to the above-described embodiments, the alerts for patients on the patient list screen 70 of the healthcare worker terminal are displayed in a color-coded manner on the basis of their types set according to the severity or urgency of patients' symptoms. This enables a healthcare worker to identify a patient with a high degree of severity or urgency in a short time.

According to the above-described embodiments, even after a healthcare worker has provided care to a patient in response to an alert, care provided information indicating that the care has been provided is displayed on the display of the healthcare worker terminal 30 for a predetermined period, rather than immediately removing the display for the patient, so the healthcare worker can monitor the condition of the patient until the condition becomes stable after providing the care to the patient.

The present disclosure is not limited to the above-described embodiments. For example, two or more blocks illustrated in the block diagram may be integrated, or one block may be divided. Instead of executing two or more steps described in the flowchart in time series according to the description, the steps may be executed in parallel or in a different order, depending on the processing capability of the device that executes each step or as necessary. In addition, modifications can be made without departing from the gist of the present disclosure.

What is claimed is:

1. A system for monitoring patients in a medical facility, the system comprising:
an interface circuit connectable to a server that stores patient information about each of the patients, the patient information including a value corresponding to each of one or more physiological parameters of the patient;
a display;
an input device;
a memory that stores a program; and
a processor configured to execute the program to:generate a first screen related to one of the patients and accepting an input of:a condition for an alert to be output depending on a value corresponding to a physiological parameter of said one of the patients, and a type of the alert,control the display to display the generated first screen, andafter a first condition for a first physiological parameter and a first type are input for a first patient via the input device, generate a second screen showing a list of the patients and capable of outputting an alert for each of the patients, control the display to display the generated second screen, acquire a first value corresponding to the first physiological parameter from the server, determine whether the first condition is met based on the first condition and the acquired first value, and upon determining that the first condition is met, update the second screen to output an alert of the first type in association with the first patient.

2. The system according to claim 1, wherein the processor executes the program to:when the alert of the first type is being output, determine whether first information indicating that care has been provided to the first patient is input via the input device, and
upon determining that the first information is input, disable the alert and update the second screen to display second information indicating that care has been provided to the first patient.

3. The system according to claim 2, wherein the processor executes the program to display the second information on the second screen for a first period and then delete the second information from the second screen.

4. The system according to claim 3, wherein the processor executes the program to determine the first period based on an attribute of the first patient.

5. The system according to claim 4, wherein the attribute includes age or disease of the first patient.

6. The system according to claim 3, wherein the processor executes the program to determine the first period corresponding to the first type.

7. The system according to claim 2, wherein the second information indicates time elapsed since the care was provided.

8. The system according to claim 2, wherein the processor executes the program to continue to display, on the second screen, third information indicating that care has not been provided to the first patient until the first information is input.

9. The system according to claim 1, wherein the alert is displayed in different colors depending on the type of the alert.

10. The system according to claim 9, wherein
the processor executes the program to display, on the second screen, the alert in a first color corresponding to the first type and the first value in the first color.

11. The system according to claim 1, wherein the first physiological parameter is one of physical information, a vital sign, and a symptom.

12. The system according to claim 1, wherein the first value is a value measurable from the first patient.

13. A system for displaying statuses of patients in a medical facility, the system comprising:
an interface circuit connectable to a server that stores patient information about each of the patients, the patient information including a value corresponding to each of one or more physiological parameters of the patient;
a display;
an input device;
a memory that stores a program; and
a processor configured to execute the program to:generate a first screen related to one of the patients and accepting an input of:a condition for an alert to be output depending on a value corresponding to a physiological parameter of said one of the patients, and a type of the alert,control the display to display the generated first screen, andafter a first condition for a first physiological parameter and a first type are input for a first patient via the input device, generate a second screen showing a list of the patients and capable of outputting an alert for each of the patients with a value of a physiological parameter related to the alert,control the display to display the generated second screen, acquire a first value corresponding to the first physiological
parameter from the server, determine whether the first condition is met based on the first condition and the acquired first value, and upon determining that the first condition is met, update the second screen to output, in association with the first patient:an alert symbol corresponding to the first type, the first value, andfirst information indicating that care has not been provided.

14. The system according to claim 13, wherein the alert symbol is displayed in different colors depending on the type of the alert.

15. The system according to claim 14, wherein the processor executes the program to display, on the second screen, the alert symbol and the first value in a first color corresponding to the first type.

16. The system according to claim 13, wherein the processor executes the program to:when the alert of the first type is being output, determine whether second information indicating that care has been provided to the first patient is input via the input device, andafter determining that the second information is input, delete the alert symbol, the first value, and the first information from the second screen.

17. The system according to claim 13, wherein the processor executes the program to, upon selection of the first patient on the second screen:generate a third screen showing a history of values of each of one or more physiological parameters of the first patient, values of the first physiological parameter being displayed in a first color corresponding to the first type, andcontrol the display to display the third screen.

18. The system according to claim 17, wherein the one or more physiological parameters are related to symptoms of dyspnea, edema, fatigue, loss of appetite, or lack of sleep.

19. A system for displaying statuses of patients in a medical facility, the system comprising:
an interface circuit connectable to a server that stores patient information about each of the patients, the patient information including a value corresponding to each of one or more physiological parameters of the patient;
a display;
an input device;
a memory that stores a program; and
a processor configured to execute the program to:generate a first screen related to one of the patients and accepting an input of:a condition for an alert to be output depending on a value corresponding to a physiological parameter of said one of the patients, and a type of the alert,control the display to display the generated first screen, andafter a first condition for a first physiological parameter and a first type for a first patient are input via the input device, generate a second screen showing a list of the patients and capable of outputting an alert for each of the patients, control the display to display the generated second screen, acquire a first value corresponding to the first physiological parameter from the server,determine whether the first condition is met based on the first condition and the acquired first value, upon determining that the first condition is met, update the second screen to output an alert of the first type in association with the first patient, and upon selection of the first patient on the second screen, generate a third screen showing a history of values of each
of one or more physiological parameters of the first patient, values of the first physiological parameter being displayed in a particular color corresponding to the first type, andcontrol the display to display the third screen.

20. The system according to claim 19, wherein the one or more physiological parameters are related to symptoms of dyspnea, edema, fatigue, loss of appetite, or lack of sleep.
